SEQUENCE LISTING (1 sequence)

SEQ ID NO: 1 - protein, 16 aa, synthetic construct
EMHKMEPCPMYRRHLG

(19) United States
(12) Patent Application Publication
(10) Pub. No.: US 2025/0041195 A1
(43) Pub. Date: Feb. 6, 2025

(54) COLLAGEN TYPE III COMPOSITION PREPARED FROM FIBROBLAST EXTRACELLULAR MATRIX

(71) Applicant: SINO UNITED (BEIJING) BIOMEDICAL TECHNOLOGY CO., LTD., Beijing (CN)

(72) Inventors: Aijie QI, Beijing (CN); Shaobo LI, Beijing (CN); Qingxuan CHEN, Beijing (CN); Subi WEI, Beijing (CN)

(73) Assignee: SINO UNITED (BEIJING) BIOMEDICAL TECHNOLOGY CO., LTD., Beijing (CN)

(21) Appl. No.: 18/923,683

(22) Filed: Oct. 23, 2024

Related U.S. Application Data
(63) Continuation of application No. PCT/CN2023/131608, filed on Nov. 14, 2023.

(30) Foreign Application Priority Data
Nov. 15, 2022 (CN) 202211422645.5

(51) Int. Cl. A61K 8/65; A61Q 19/08; C07K 7/08; C07K 14/78; C12N 5/077

(52) U.S. Cl. CPC A61K 8/65; A61Q 19/08; C07K 7/08; C07K 14/78; C12N 5/0656; A61K 2800/805; A61K 2800/85; C12N 2500/02; C12N 2523/00

(57) ABSTRACT

The present application provides a collagen type III composition prepared from a fibroblast extracellular matrix. The collagen type III is ultimately prepared by further purification, detection, and isolation based on the extracellular matrix components in a conditioned culture solution for fibroblasts, which are the most important cells in the human skin dermis, and can be combined with hyaluronic acid to form a composition formulation for use by people who want to remove and fill wrinkles on face and neck. The method is a production technique of the collagen type III composition from the fibroblast extracellular matrix, which belongs to a transplantation technology. More specifically, the human collagen is prepared from the cell culture supernatant by in vitro culturing the skin donated by volunteers for fibroblasts. The detection results of HIV, HBV, HCV, and syphilis infection markers are all non-reactive, mycoplasma is negative, and bacterial detection is negative.

### CROSS-REFERENCE TO RELATED APPLICATIONS

This application is a continuation of international PCT application serial no. PCT/CN2023/131608, filed on Nov. 14, 2023, which claims the priority benefit of China application serial no. 202211422645.5, filed on Nov. 15, 2022 and entitled “Collagen Type III Composition Prepared from Fibroblast Extracellular Matrix”. The entirety of each of the above-mentioned patent applications is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Oct. 15, 2024, is named 148788-0C-us-sequence listing and is 1,840 bytes in size.

### BACKGROUND OF THE INVENTION

### 1. Technical Field

The present application relates to the field of biology, and more particularly, to a collagen type III composition prepared from fibroblast extracellular matrix.

### 2. Description of Related Art

Cell therapy is currently one of the hot spots in the development of biomedical therapy worldwide. Due to the low immunogenicity, hematopoietic stem cell transplantation or autologous somatic cell or stem cell therapy is the earliest therapeutic regimen for clinical application at home and abroad. FDA of the United States approved autologous fibroblasts for clinical application over a decade ago. For more than a decade, there have been thousands of cases of autologous fibroblast transplantation, and fruitful results have been achieved in clinical application. During the process of in vitro culture, expansion, and preparation of a cell suspension of fibroblasts, a large amount of cell culture supernatant containing a large amount of human fibroblast extracellular matrix will be produced. There are no good reports on the study of extracellular matrix.

The extracellular matrix is mainly composed of five kinds of substances, namely collagen, non-collagen, elastin, proteoglycan, and aminoglycan, and is mainly classified into basement membrane and interstitial matrix according to the distribution site. Collagen, an insoluble fibrous protein, is a major component of the extracellular matrix and is distributed throughout various organs and tissues. Collagen generally accounts for 25% (mass fraction) of the total amount of proteins in mammals. Collagen in connective tissues is mainly type I, II, and III; type IV collagen is mainly present in the basement membrane. Collagen provides the ability to resist external tension for the animal's connective tissues. The basic structure is a triple helix structure formed by three intertwining collagen polypeptide chains, with a diameter of 1.5 nm. Some types of collagen triple helixes can be combined into an ordered polymer in which the collagen triple helixes are parallel to each other. This ordered polymer is called collagen fibril, and has a diameter of 10-300 nm and a length of up to several microns. The collagen fibrils can be further assembled into a collagen fibre with a diameter of 0.5-3 μm and a length of hundreds of microns, which is one of the main components of the extracellular matrix. The orientation of the collagen fibre is controlled by fibroblasts. In tendon tissue, for example, fibroblasts can pull the collagen fibres to move directionally, so that they are distributed along the axis of tension in the tissue. Other types of collagens function to modify the surface of collagen fibres to facilitate the connection between collagen fibres and between collagen fibres and other components in the extracellular matrix, such as glycoproteins. The common feature of non-collagen glycoproteins is that they can bind to both cells and other macromolecules in the extracellular matrix, thereby adhering cells to the extracellular matrix. The glycoproteins in ECM include adhesion proteins such as laminin (LN), fibronectin (FN), contactin (ND), and anti-adhesion proteins such as tenascin (TN), osteonectin (BM-40), basement membrane protein (Bamin), etc. TN is an oligoglycoprotein in ECM, and BM-40 is a cysteine-rich ECM glycoprotein. Proteoglycans and aminoglycans, also known as proteoglycans or mucopolysaccharides, are glycoproteins. Their sugar chains are mostly long-chain aminopolysaccharides, and many long-chain aminopolysaccharides are linked to a protein core to form glycoproteins. They are called proteoglycans because the content of saccharides in them is much more than that of proteins, sometimes the content of saccharides being up to 95%. Proteoglycans act as a major component of connective tissues. The aminopolysaccharides constituting proteoglycans include hyaluronic acid, chondroitin and chondroitin sulfate, dermatan sulfate, keratin sulfate, heparin and heparan sulfate, etc. They are all negatively charged glycans with carboxyl or sulfate groups in structure. These glycans, except for hyaluronic acid, are all chain-linked to a core protein molecule. The linked glycans may be a single chain or multiple chains.

Recently, a study by the University of Michigan in the United States has shown that the increase of extracellular matrix can delay aging. The extracellular matrix components can be introduced by filling. For hyaluronic acid injection, for example, the extracellular matrix content is increased by directly filling hyaluronic acid into the dermis. However, a single polysaccharide component can only maintain physical support for a period of time, but is not enough to induce extracellular matrix synthesis by itself. The recent hot Sculptra is to introduce polylactic acid microspheres into the dermis, which, on one hand, provides mechanical support, and on the other hand, induces local inflammation to stimulate the synthesis of extracellular matrix. However, inflammatory stimulation is often difficult to control and often causes strong side effects. In summary, the current ideal approach is to supplement the extracellular matrix directly. Compared with the introduction of a single component, the introduction of the extracellular matrix as a whole can better simulate the actual skin condition. The extracellular matrix can not only provide mechanical support, but also play a role in regeneration. The extracellular matrix has a plurality of components that cooperate with each other to remodel the microenvironment in the dermis. By regulating the skin microenvironment, on the one hand, the components (collagen, elastin, etc.) of the matrix are restored to a healthy proportion, structure, and function; on the other hand, the balance of matrix synthesis/degradation is reconstructed. At present, the domestic feasible technical solutions are still relatively few and need further optimization.

### BRIEF SUMMARY OF THE INVENTION

To meet the requirements of the prior art, the present application establishes a technical system for preparing a large amount of fibroblast extracellular matrix from the in vitro culture supernatant of human fibroblasts and then isolating and extracting collagen type III through years of experimental studies.

In an aspect, the method is a production technique of type III/ternary collagen composition from a fibroblast extracellular matrix. More specifically, the skin donated by volunteers, the skin aseptically cut alone, or the skin discarded or deliberately cut by surgical procedures such as eye pouch resection, double eyelid operation, and wrinkle removal, etc., was used as tissues for dermal fibroblasts culturing in vitro under a simulated human body environment, a fibroblast extracellular matrix concentrate was prepared from the cell culture supernatant, and detected by SDS-polyacrylamide gel electrophoresis. The molecular weight of the collagen extract is between 45 KD and 75 KD. The detection results of HIV, HBV, HCV, and syphilis infection markers are all non-reactive; mycoplasma is negative, and bacterial detection is negative. The type III collagen extracted and isolated from the fibroblast extracellular matrix may be further combined with hyaluronic acid in different ratios to prepare cosmetics, drugs, and medical devices such as medical dermal fillers.

In another aspect, an active polypeptide with high activity is provided to promote the proliferation of fibroblasts and the secretion of extracellular matrix, which can effectively promote the proliferation of fibroblasts and the secretion of extracellular matrix.

Further, the sequence of the active polypeptide of the present application is shown in SEQ ID NO: 1 (i.e., EMHKMEPCPMYRRHLG). The active polypeptide is obtained by screening from a polypeptide library based on a fibroblast model. The active polypeptide has the characteristics of strongly stimulating the proliferation of fibroblasts while promoting the secretion of extracellular matrix and collagen.

Further, the N-terminus of the active polypeptide is acetylated or the C-terminus thereof is amidated, or both the N-terminus and the C-terminus thereof are PEG-modified.

The polypeptide can be prepared using the Fmoc polypeptide synthesis method.

For ease of illustration, the present application designates the above polypeptide as HXT-32.

The Fmoc polypeptide synthesis method is a conventional method used by a person skilled in the art to prepare a polypeptide. In the case that a person skilled in the art knows that the polypeptide to be prepared needs to comprise an amino acid sequence as shown in EMHKMEPCPMYRRHLG, in combination with the existing Fmoc polypeptide synthesis method, the polypeptide to be protected in the present application may be obtained. The duration of the polypeptide in the general chronic toxicology study in mice is ≥6 months, following the guidelines of ICH S4, demonstrating that it has no significant toxic side effects on mice.

Further, the polypeptide of the present application can also be prepared and obtained through biotechnology and prokaryotic expression.

Further, the type III collagen extracted and isolated from the fibroblast extracellular matrix may be combined with hyaluronic acid and the active polypeptide in different ratios to prepare a composition with higher activity, which can not only work on its own, but also can accelerate the secretion of active substances such as collagen from the skin, thereby contributing to skin anti-aging.

Further, the fibroblasts of the present application can be prepared by the following methods:
1. Routine serological tests are performed for HIV, HBV, HCV, and syphilis before volunteers donate their skin. If the results are all negative, the cell culture supernatant is retained for subsequent preparation of fibroblast extracellular matrix, and if positive, it is discarded;
2. The skin aseptically cut alone, or the skin discarded or deliberately retained by surgical procedures such as eye pouch resection, double eyelid operation, and wrinkle removal, etc., is used as tissues for cell culture in vitro;
3. Primary fibroblasts are cultured at 37° C. in a 5-10% CO₂ incubator using conventional tissue blocks or enzymatic digestion methods, takes either high sugar DMEM or DMEM/F12 (1:1), supplemented with 5-10% fetal bovine serum, or a serum-free culture medium as the basic culture medium.

Further, the extracellular matrix of the present application can be prepared by culturing the fibroblasts prepared according to the present application in DMEM/F12 containing 200 μg/mL the active polypeptide and 10% fetal bovine serum for 24 h, collecting the cell culture supernatant, sterilizing the collected cell culture supernatant by filtering it with a 0.22 μm filter membrane to obtain a sterile cell culture solution; concentrating the extracellular matrix using an ultrafiltration system with an ultrafiltration membrane with a molecular weight of ≤below 90 KD to obtain the fibroblast extracellular matrix.

Further, the obtained fibroblast extracellular matrix is subjected to viral inactivation by radiation sterilization and/or heating. The optimum conditions for radiation sterilization are as follows: 60Co-γ ray of 25 KGy, a heating temperature of 55±0.5° C., and a continuous duration of about 8-10 hours.

Further, the fibroblast extracellular matrix is combined with hyaluronic acid and the active polypeptide at an optimized mass ratio of 7.3:2.5:0.2 to prepare a collagen type III composition.

Further, the present application provides use of the collagen type III composition in the preparation of drugs for resisting skin aging.

Further, the present application provides use of the collagen type III composition in the preparation of tissue engineering medical devices and bio-innovative drugs.

Further, the present application provides use of the collagen type III composition in the preparation of cosmetics for resisting skin wrinkles.

Further, the cosmetics of the present application may be skincare cosmetics such as lotions, emulsions, creams, and facial masks; body cosmetics; makeup removal products such as cleansing oils; skin cosmetics such as bath gels and hand sanitizers; hair cosmetics such as hair liquids, hair oils, hair tonics, hair growth agents; and the like.

Although the additive amount of the composition in the cosmetics of the present application is not particularly limited, it is usually blended in the cosmetics by 10-50% by weight, preferably by 20-50% by weight.

Various components commonly used in cosmetics, drugs, and the like may be further blended into the cosmetics of the present application as needed and within the scope of not impairing the effects of the present application. For example, humectants, hydrocarbons, higher alcohols, higher fatty acids and their triglycerides, ester oils, animal and vegetable fats, silicones, vitamins, UV absorbers, water-soluble polymers, antioxidants, cationic surfactants, anionic surfactants, amphoteric surfactants, nonionic surfactants, sequestrants, alcohols, thickening agents, preservatives, coloring matters, pigments, perfumes, etc. may be cited.

### Beneficial Effects

The present application provides a method for promoting the proliferation of fibroblasts through the screened active polypeptide, and at the same time, the corresponding fibroblast extracellular matrix is prepared and obtained. The extracellular matrix extracted and isolated from the fibroblast culture solution may be combined with hyaluronic acid and the active polypeptide in different ratios to prepare a composition with higher activity, which can not only work on its own, but also can accelerate the secretion of active substances such as collagen from the skin, thereby contributing to skin anti-aging. The composition may also be prepared into cosmetics, drugs, and medical devices such as medical dermal fillers.

### BRIEF DESCRIPTION OF THE SEVERAL VIEWS OF THE DRAWINGS

One or more examples are illustrated with reference to corresponding figures in accompanying drawings, which do not impose a limitation on the examples. The specialized word “exemplary” herein means “serving as an instance, example, or illustration”. Any example illustrated herein as “exemplary” is not necessarily to be construed as preferred or advantageous over other examples.

FIG. 1 is an image of fibroblasts.

FIG. 2 shows the effect of the active polypeptide on the viability of human fibroblasts.

FIG. 3 shows the effect of the active polypeptide on the secretion of collagen from human fibroblasts.

### DETAILED DESCRIPTION OF THE INVENTION

The present application will be described in detail hereinafter with reference to specific embodiments and examples, from which advantages and various effects of the present application will become more apparent. It should be understood by those skilled in the art that these specific embodiments and examples are intended to illustrate rather than limit the present application. Throughout the specification, terms as used herein should be understood to have the meanings commonly used in the art unless otherwise indicated. Accordingly, unless defined otherwise, all technical and scientific terms as used herein have the same meaning as generally understood by one of ordinary skill in the art to which the present application belongs. In case of conflict, the specification shall control. Unless otherwise specified, various raw materials, reagents, instruments, devices, and the like used in the present application are commercially available or can be prepared by existing methods.

### Example 1. Preparation of Human Skin Fibroblasts

### Fibroblast Preparation:

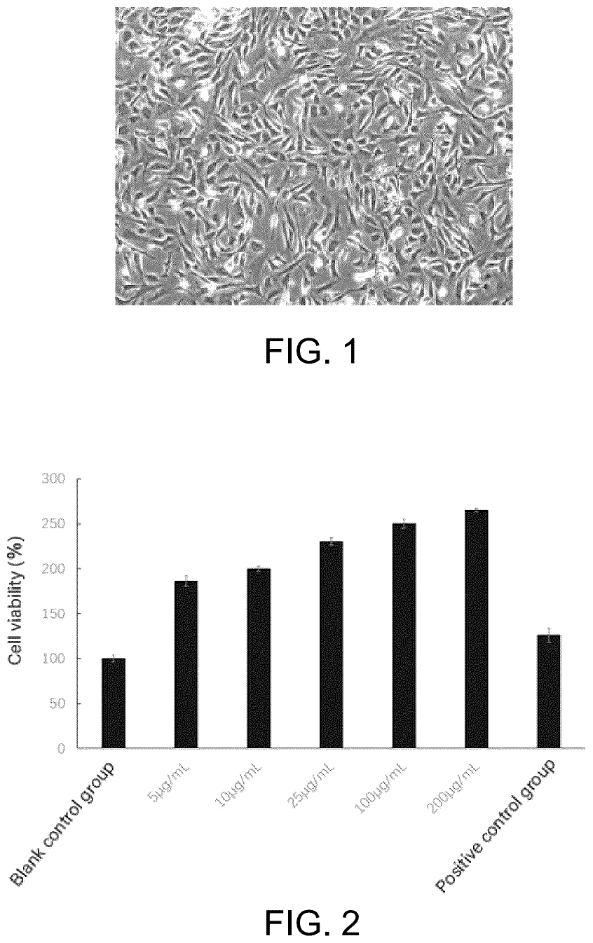

1. Main reagents: DMEM/F12 (Gibco); Superior fetal bovine serum; Dispase II (Roche); Trypsin (Sigma); Triton X100 (Sigma); Mouse anti-human vimentin monoclonal antibody (Santa); Rabbit anti-human cytokeratin 15 monoclonal antibody (Abcam); Immunohistochemistry SP kit (Maxin); DAB chromogenic solution (Maxin); FITC-labeled anti-mouse and anti-rabbit IgGs (Beijing Zhongshan Jinqiao Biotechnology Co., LTD).
2.1 Primary culture: human foreskin skin tissue was taken and rinsed 3 times with PBS containing penicillin-streptomycin to remove subcutaneous connective tissue as much as possible. The skin was cut into small blocks and plated on a dish with the dermis side down, to which Dispase II was added and incubated at 4° C. overnight. The epidermis and dermis were isolated on the next day. The dermis was cut into small blocks, inoculated in a 35 mm culture dish, and cultured at 37° C. in a 5% CO₂ constant temperature incubator for 1 h. A small amount of DMEM/F12 (3:1) containing 10% superior fetal bovine serum was added. A sufficient amount of culture medium was added on the next day for further culture and then changed every 3 days.
2.2 Passage: the cells were digested with 0.25% (mass fraction) trypsin-0.02% EDTA for about 1 min. After observing the cells shrinking and rounding and the intercellular space increasing under a microscope, DMEM/F12 (3:1) containing 10% superior fetal bovine serum was added to stop the digestion. The cells were pipetted up and down and passaged at a ratio of 1:3.
2.3 Harvesting of cells: after passaged for 4-6 generations, cells were harvested and washed 3 times with normal saline, and 5×10⁷ fibroblasts were suspended in 1 ml of normal saline for later use.
3. Identification
3.1 Morphological observation: the morphology of cells in different culture stages was observed using an inverted phase-contrast microscope. After 3 days of primary culture, cells were seen growing around the tissue blocks. These cells were spindle-shaped and grew like fibrocytes. On the 7th to 10th day, a large number of cells can be seen growing, like a school of fish. See FIG. 1. The hFbs in passage culture showed vigorous growth and reached confluence at 3-4 days. Their morphology was similar to that of the primary cells and tended to be more consistent. The cells were long spindle-shaped, and the nuclei were oval-shaped with 1-2 nucleoli.
3.2 Immunocytochemistry: P6 generation cells growing on a glass slide were taken, washed 3 times with PBS, fixed with 4% paraformaldehyde at room temperature for 20 min; then washed with PBS, incubated with 0.1% Triton X100 for 10 min; blocked with 3% hydrogen peroxide at room temperature for 20 min; then washed with PBS, covered with serum for 20 min; incubated with mouse anti-human vimentin primary antibody and rabbit anti-human cytokeratin (CK) 15 primary antibody at 1:100 dilution at 4° C. overnight; then washed with PBS, and incubated with secondary antibody at room temperature for 30 min. After washing with PBS, DAB was added, and the cells were re-stained with hematoxylin. Moreover, PBS was used as a negative control instead of the primary antibody. The cells were observed under a microscope and photographed. The immunocytochemical results showed that the cytoplasm of P6 generation hFbs was stained dark brown, vimentin was positive, and CK15 was negative.
3.3 Flow cytometry: P3 and P6 generation cells were digested with 0.25% trypsin and 0.02% EDTA and collected with 2×10⁵ cells/sample. The cells were washed with PBS, incubated with 0.1% Triton X100 at room temperature for 10 min; then washed with PBS, incubated with mouse anti-human vimentin primary antibody and rabbit anti-human CK15 primary antibody at 1:50 dilution at room temperature for 30 min; then washed with PBS, and incubated with FITC-labeled anti-mouse and anti-rabbit IgGs at 1:50 dilution as secondary antibody at 4° C. for 30 min in the dark. The obtained cells were washed twice with PBS and resuspended in 400 μl of PBS for FACS analysis. The markers of hFbs were detected by flow cytometry. After more than 3 passages, the cells were homogeneous, and vimentin-positive cells accounted for more than 95% of the total number; CK15-positive cells accounted for less than 2% of the total number, which was regarded as negative expression and considered qualified.

### Example 2. Effect of the Active Polypeptide on the Viability of Human Fibroblasts

The active polypeptide of the present application with a sequence of EMHKMEPCPMYRRHLG was serially diluted in a 96-well cell culture plate to 5, 10, 25, 50, 100, 200 μg/mL, with 5 duplicated wells per concentration. The above operations were all performed under sterile conditions.

For the passage of the cell lines isolated in Example 1, the cell culture medium was firstly poured out, and the cells were washed twice with 2 mL of PBS. The liquid was discarded, and 1 mL of 0.25% (mass fraction) trypsin solution was added for digestion for 3 min until the cells were completely detached. The cell suspension was transferred to a centrifuge tube, 2 mL of DMEM/F12 containing 10% superior fetal bovine serum was added to stop the digestion, and then centrifugation was performed at 1000 r/min for 5 min. After the completion of centrifugation, the supernatant was discarded, and 2 mL of DMEM/F12 containing 10% superior fetal bovine serum was added and fully pipetted up and down. The cell solution was adjusted to a concentration of 5.0×10⁵ cells/mL and cultured at 37° C. under 5% carbon dioxide and saturated humidity conditions. After 24 h of passaging, the cells were used for pharmacological activity assay. The above operations were all performed under sterile conditions.

The cells were inoculated into a 96-well cell culture plate, with 100 μL per well, and incubated at 37° C. under 5% carbon dioxide and saturated humidity conditions. After 24 h, the culture medium was changed to a maintenance medium, and then the cells were further cultured at 37° C. under 5% carbon dioxide and saturated humidity conditions for 24 h. The maintenance medium in the prepared cell culture plate was discarded. A solution of the active polypeptide was added at 100 μL per well. The blank control group (only 100 μL of the maintenance medium was added) and the positive control group (100 μL of 10 ng/mL bFGF maintenance medium was added) were set. The cells of each group were cultured at 37° C. under 5% carbon dioxide and saturated humidity conditions for 48 h. Then, MTT solution was added to the cell culture at 10 μL each well, and the cells were incubated at 37° C. under 5% carbon dioxide and saturated humidity conditions for 5 h. The above operations were all performed under sterile conditions. After discarding the liquid in the culture plate, 100 μL of dimethyl sulfoxide was added to each well and mixed well. The absorbance was determined at 570 nm by a microplate reader, with 630 nm as a reference wavelength. The results were recorded and the cell viability was calculated according to the following formula: cell viability=(absorbance A570 of the experimental group/absorbance A570 of the blank control group)×100%. The results were shown in FIG. 2.

As can be seen from FIG. 2, the effect of the active polypeptide for promoting the survival of human fibroblasts was gradually enhanced in a dose-dependent manner with increasing concentration of the active polypeptide in the concentration range of 0-200 μg/mL. When the mass concentration of the active polypeptide was 200 μg/mL, it could significantly promote the growth of fibroblasts, and the cell viability was 265.3%±4.52%, which was much higher than that of the control group. * indicates P<0.05 compared with the blank control group, which means that the difference is significant.

### Example 3. Effect of the Active Polypeptide on the Secretion of Collagen from Human Fibroblasts

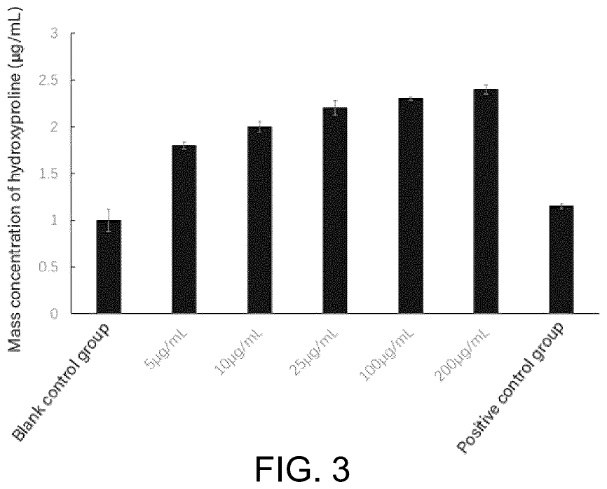

Because hydroxyproline is relatively high in collagen, the amount of hydroxyproline can reflect the metabolism of collagen. The hydroxyproline content was determined using a hydroxyproline content assay kit (Cat. No: AKAM017). The cells of each group cultured in Example 2 were quantified to the same density for assay. The assay results were shown in FIG. 3.

As can be seen from FIG. 3, the production of hydroxyproline was gradually increased in a dose-dependent manner with increasing concentration of the active polypeptide in the concentration range of 0-200 g/mL. When the mass concentration of the active polypeptide was 200 μg/mL, it could significantly promote the secretion of collagen from fibroblasts, and the hydroxyproline content was (2.42±0.05) μg/mL, which was much higher than that of the control group. * indicates P<0.05 compared with the blank control group, which means that the difference is significant.

### Example 4. Collagen Type III Composition Prepared from a Fibroblast Extracellular Matrix and the Activity Verification Thereof

The fibroblasts prepared in Example 1 were cultured in DMEM/F12 containing 200 μg/mL of the active polypeptide and 10% fetal bovine serum for 24 h. The cell culture supernatant was collected, and sterilized by filtering it with a 0.22 μm filter membrane to obtain a sterile cell culture solution. The extracellular matrix was concentrated using an ultrafiltration system with an ultrafiltration membrane with a molecular weight of ≤90 KD to obtain the fibroblast extracellular matrix. The obtained fibroblast extracellular matrix was subjected to viral inactivation by radiation sterilization. The condition for radiation sterilization was 60Co-γ ray of 25 Kgy. The obtained fibroblast extracellular matrix was detected by SDS-polyacrylamide gel electrophoresis. The molecular weight of the fibroblast extracellular matrix was between 45 KD and 75 KD, indicating that collagen type III was isolated and prepared. The results were substantially consistent with those obtained by other isolation methods in the prior art.

The fibroblast extracellular matrix (also known as collagen type III) was combined with hyaluronic acid and the active polypeptide at an optimized mass ratio of 7.3:2.5:0.2 to prepare a collagen type III composition.

The fibroblast extracellular matrix (also known as collagen type III) was combined with hyaluronic acid at an optimized mass ratio of 7.5:2.5 to prepare a binary collagen composition.

Sixty healthy subjects were selected. Inclusion criteria: (1) Healthy women aged 35-60 years; (2) Clear fine lines at the corners of eyes; (3) Lack of elasticity in the skin around the corners of eyes; clinical score of the wrinkle appearance (visual inspection) of “crows feet”≥4 [Clinical score was determined according to Skin Aging Atlas (Volume 2 Asian Type)]; (3) Have ability to well cooperate with the tester and keep the regularity of life during the study; (4) Have ability to read and understand all the contents of the informed consent form, and volunteer to sign the informed consent form; (5) Discontinue use of skin care products during the test; (6) No longer participate in the clinical trial of any other study site during the test; (7) Approve not to use any cosmetics, drugs, and health products that have an influence on the results during the test. Exclusion criteria: subjects who meet any of the following conditions are excluded from the study: (1) Patients with skin diseases at the test site that may affect the determination of test results; (2) Hyperallergic constitution; (3) Female patients who are pregnant, lactating or intend to become pregnant during the test; (4) Patients with severe heart, liver, and kidney damage and severe immune dysfunction; (5) Patients with mental diseases, severe endocrine diseases, and oral contraceptives; (6) Patients who have participated in drug clinical test or other tests within 30 days, or patients who have systematically used drugs that have an impact on test results within the last 1 week; (7) Patients who have oral and topical cosmetic products that may affect the test results within 2 weeks; (8) Unable to cooperate with the tester; (9) Patients considered unsuitable for participating in the study by the investigator.

Subjects were randomized into three groups: a collagen type III composition group, a binary collagen composition group, and a blank group, with 20 cases in each group. The experimental period was 10 weeks. For the product groups, the same amount of the compositions was applied to the corner of the eyes during the experiment, twice a day, once in the morning and once in the evening, for 10 consecutive weeks. For the blank group, only basic moisturizing cosmetics were used during the experiment, without using any cosmetics or drugs with anti-wrinkle efficacy. Subjects were followed up at week 0 before use and at week 10 after use, respectively, with each follow-up being at the same time during the day. VISIA-CR was used to collect the images of subjects, and the changes in eye wrinkles and textures before and after using the composite anti-wrinkle eye cream were compared. A rapid optical imaging system for skin, Primos, was used to detect skin wrinkles. The lower the Sa value, the fewer skin wrinkles. The results were shown in Table 1.

TABLE 1
Sa values of skin wrinkles

| | Sa value | Sa value |
|---|---|---|
| Group name | at week 0 | at week 10 |
| Collagen type III composition group | 33.84 ± 0.30 | 30.13 ± 0.12* |
| Binary collagen composition group | 33.76 ± 0.19 | 31.83 ± 0.14* |
| Blank group | 33.52 ± 0.23 | 34.67 ± 0.25 |

As can be seen from Table 1, the results of the subjects in the composition groups and in the blank group showed that there was no significant difference between the Sa values of the two groups before using the compositions; after 10 weeks of use of the composition, the Sa values of skin wrinkles for both the binary collagen composition and the collagen type III composition group showed a significant reduction trend, and the Sa value of skin wrinkles for the composition groups significantly decreased (* indicates P<0.05). The Sa value of skin wrinkles for the blank group increased after 10 weeks, but there was no significant difference as compared with that at week 0. The lower the Sa value, the fewer skin wrinkles. It can be seen from the above data that when compared with the binary collagen composition group, the collagen type III composition group which further comprises the active polypeptide can further enhance the activity of skin fibroblasts and promote the production of collagen, which is more beneficial to anti-wrinkles, resulting in a decrease in the Sa value of eye wrinkles. Therefore, the test demonstrated that the active polypeptide, particularly the collagen type III composition containing the active polypeptide, has the effect of improving eye wrinkles and reducing the formation of wrinkles. After 10 weeks of use of the composition, the mean clinical scores of conjunctiva, iris, cornea, and other injuries in subjects were all 0, indicating that the composite composition has a good safety.

The collagen type III composition can be packaged in different doses, combined with hyaluronic acid in different ratios as needed, and then added to other drugs, medical device implants, and cosmetics for use.

Finally, it is also noted that the terms “include”, “comprise”, or any other variation thereof are intended to cover a non-exclusive inclusion, such that a process, method, article, or device including a list of elements includes not only those elements but also other elements not explicitly listed, or elements inherent to such a process, method, article, or device. Although preferred examples of the present application have been described, a person skilled in the art can make additional changes and modifications to these examples once they learn the basic inventive concept. Therefore, the appended claims are intended to be construed as to cover the preferred examples and all changes and modifications falling within the scope of the present application. Obviously, a person skilled in the art can make various modifications and variations to the present application without departing from the spirit and scope of the present application. Thus, the present application is intended to cover these modifications and variations if they fall within the scope of the claims and their equivalents of the present application.

### INDUSTRIAL APPLICABILITY

The collagen type III composition prepared from the fibroblast extracellular matrix provided in the present application not only has high activity on its own but also can accelerate the secretion of active substances such as collagen from the skin, which is beneficial to skin anti-aging. The composition may also be used to prepare cosmetics, drugs, and medical devices such as medical dermal fillers, and it has a good potential for application.

What is claimed is:

1. An active polypeptide having an amino acid sequence shown in SEQ ID NO:1.

2. A collagen type III composition prepared from a fibroblast extracellular matrix, which is prepared by a method of: culturing human skin fibroblasts in a culture medium containing the active polypeptide according to claim 1, collecting the cell culture supernatant, filtering the collected cell culture supernatant through a filter membrane to obtain a sterile cell culture solution; concentrating the extracellular matrix using an ultrafiltration system with an ultrafiltration membrane with a molecular weight cut-off of ≤90 KD to obtain the fibroblast extracellular matrix; performing viral inactivation by radiation sterilization to obtain collagen type III with a molecular weight between 45 KD and 75 KD; and combining the collagen type III with hyaluronic acid and the active polypeptide to prepare the collagen type III composition.

3. The collagen type III composition according to claim 2, wherein the culture medium contains 200 μg/mL of the active polypeptide;
and/or, the culture medium is DMEM or DMEM/F12 containing 5%-10% fetal bovine serum, or a serum free culture medium;
and/or, the culture of human skin fibroblasts is conducted for 24 h.

4. The collagen type III composition according to claim 2, wherein the filter membrane has a pore size of 0.22 μm;
and/or, the radiation sterilization is performed under a condition of 60Co-γ ray of 25 Kgy.

5. The collagen type III composition according to claim 2, wherein the collagen type III is combined with hyaluronic acid and the active polypeptide at a mass ratio of 7.3:2.5:0.2.

6. The collagen type III composition according to claim 2, wherein the human skin fibroblasts are isolated and prepared from the skin, and the human refers to a healthy person having no blood-borne diseases such as hepatitis B, hepatitis C, syphilis, and AIDS through hematological examination.

7. The collagen type III composition according to claim 6, wherein the fibroblast extracellular matrix is prepared by a method of:
a) obtaining human skin fibroblasts from skin tissue using tissue blocks or digestion methods;
b) expanding the human skin fibroblasts on a large scale in vitro at 37° C. under 5-10% CO₂ in a culture medium containing fetal bovine serum or a serum-free culture medium;
c) obtaining a large amount of cell culture supernatant while expanding the cells;
d) concentrating the culture supernatant by ultrafiltration using an ultrafiltration membrane with a molecular weight cut-off of ≤90 KD.

8. The collagen type III composition according to claim 7, wherein the fibroblast extracellular matrix concentrate obtained by ultrafiltration is heated to inactivate residual blood infectious pathogens.

9. The collagen type III composition according to claim 8, wherein the inactivated collagen type III is filtered with a 0.22 μm microporous filter membrane to obtain a sterile collagen type III.

10. A method for preventing skin wrinkles, comprise a step of use of the collagen type III composition according to claim 2.

11. A method for promoting the proliferation of human skin fibroblasts and the secretion of collagen, comprising a step of use of the active polypeptide according to claim 1.
